(19) United States
(12) Patent Application Publication
(10) Pub. No.: US 2020/0064658 A1
(43) Pub. Date: Feb. 27, 2020

(54) LOAD BALANCING OPHTHALMIC OPERATIONS METHOD AND SYSTEM

(71) Applicant: Johnson & Johnson Vision Care, Inc., Jacksonville, FL (US)

(72) Inventors: Scott Humphreys, Greensboro, NC (US); Donald K. Whitney, Melbourne, FL (US); Adam Toner, Jacksonville, FL (US)

(21) Appl. No.: 16/112,627

(22) Filed: Aug. 24, 2018

(51) Int. Cl. G02C 7/04; G06F 9/50; G02C 7/08; A61F 2/16; A61B 3/113; A61B 5/00

(52) U.S. Cl. CPC G02C 7/04; G06F 9/505; G02C 7/081; A61B 5/7282; A61B 3/113; A61B 5/4836; A61B 5/6821; A61F 2/16

(57) ABSTRACT

A method including transmitting, by a first processor disposed in or on a first ophthalmic device, first data to a second processor disposed in or on a second ophthalmic device; transmitting, by the second processor, second data to the first processor; determining, by the first processor and during a time period, a first characteristic of a user based on at least the second data; and determining, by the second processor and during the time period, a second characteristic of the user based on at least the first data.

### TECHNICAL FIELD

The present disclosure relates to ophthalmic devices having embedded controlling elements, and more specifically, to the embedded controlling elements and method for using the same to balance energy load between wearable ophthalmic devices.

### BACKGROUND

Near and far vision needs exist for all. In young non-presbyopic patients, the normal human crystalline lens has the ability to accommodate both near and far vision needs and those viewing items are in focus. As one ages, the vision is compromised due to a decreasing ability to accommodate as one ages. This is called presbyopia.

Adaptive optics/powered lens products are positioned to address this and restore the ability to see items in focus. But, what is required is knowing when to “activate/actuate” the optical power change. While a manual indication or use of a key fob to signal when a power change is required is one way to accomplish this change. However, leveraging anatomical/biological conditions/signals may be more responsive, more user friendly and potentially more “natural” and thus more pleasant.

A number of things happen when we change our gaze from far to near. Our pupil size changes, our line of sight from each eye converge in the nasal direction coupled with a somewhat downward component as well. However, to sense/measure these items are difficult, one also needs to filter out certain other conditions or noise, (e.g., blinking, what to do when one is lying down, or head movements).

In reference to FIG. 4, when observing an object in each eye the visual axis points toward the object or Target. Since the two eyes are spaced apart (distance b) and the focal point is in front, a triangle is formed. Forming a triangle allows the relationship of angles (θL and θR) of each visual axis to the distance (Y) the object is from the eyes to be determined. Since the distance (Y) is what determines if a change in optical power is required, then knowing the angles and the distance between the eyes and using simple math would allow a system to make a decision regarding when to change the optical power.

Sensing of multiple items may be required to remove/mitigate any false positive conditions that would indicate a power change is required when that is not the case. Use of an algorithm may be helpful. Additionally, threshold levels may vary from patient to patient, thus some form of calibration will likely be required as well.

A user may use multiple ophthalmic devices, such as one for each eye. However, if load is not properly balanced between the ophthalmic devices, then one ophthalmic device may lose power before the other. Thus, there is a need for more sophisticated ophthalmic devices that balance processing and communication load to prevent one device from losing power before the others.

### SUMMARY

A system of the present disclosure comprises a first ophthalmic device configured to be disposed adjacent an eye of a user, and a first sensor system disposed in or on the first ophthalmic device, the first sensor system comprising a first sensor and a first processor operably connected to the first sensor and configured to alternate between a primary mode and a secondary mode; during the primary mode, the first processor is configured to receive first data from one or more of the first sensor and a second sensor system disposed in a second ophthalmic device, determine a first characteristic of the user based on at least the first data, and transmit the first characteristic of the user to the second sensor system; and during the secondary mode, the first processor is configured to transmit second data to the second sensor system and receive a second characteristic of the user from the second sensor system, wherein the second sensor system determines the second characteristic of the user based on at least the second data.

According to another aspect of the present disclosure, a system including a first ophthalmic device configured to be disposed adjacent a first eye of a user, the first ophthalmic device comprising a first sensor system, the first sensor system comprising a first sensor and a first processor operably connected to the first sensor; a second ophthalmic device configured to be disposed adjacent a second eye of the user, the second ophthalmic device comprising a second sensor system, the second sensor system comprising a second sensor and a second processor operably connected to the second sensor; the first processor is configured to receive first data from the second sensor system and determine a first characteristic of the user during a time period based on at least the first data; and the second processor is configured to receive second data from the first sensor system and determine a second characteristic of the user during the time period based on at least the second data.

According to another aspect of the present disclosure, a method including transmitting, by a first processor disposed in or on a first ophthalmic device, first data to a second processor disposed in or on a second ophthalmic device; transmitting, by the second processor, second data to the first processor; determining, by the first processor and during a time period, a first characteristic of a user based on at least the second data; and determining, by the second processor and during the time period, a second characteristic of the user based on at least the first data.

### BRIEF DESCRIPTION OF THE OF THE DRAWINGS

FIG. 1 shows an exemplary implementation according to an embodiment of the present disclosure.

FIG. 2 shows a flowchart according to an embodiment of the present disclosure.

FIG. 3 shows another exemplary implementation according to an embodiment of the present disclosure.

FIG. 4 shows an example of focus determination.

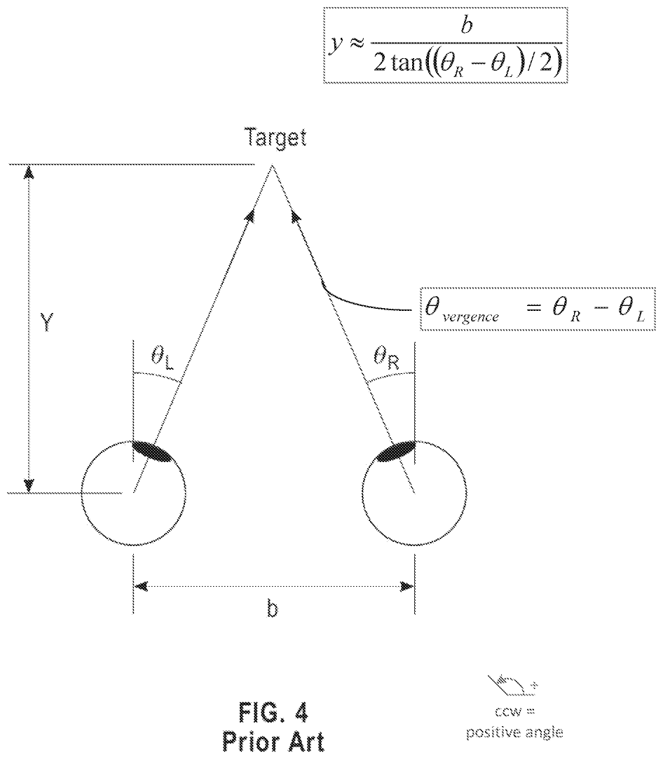

FIG. 5 shows another flowchart according to an embodiment of the present disclosure.

FIG. 6 illustrates a flow diagram according to aspects of the present disclosure

### DETAILED DESCRIPTION

Before explaining at least one embodiment of the disclosure in detail, it is to be understood that the disclosure is not limited in its application to the details of construction and the arrangement of the components set forth in the following description or illustrated in the drawings. The disclosure is applicable to other embodiments or of being practiced or carried out in various ways. Also, it is to be understood that the phraseology and terminology employed herein is for the purpose of description and should not be regarded as limiting. As will be appreciated by one skilled in the art, aspects of the present disclosure may be embodied as a system, method or computer program product.

The present methods and systems relate to an ophthalmic system comprising one or more ophthalmic devices, such as a system comprising at least one ophthalmic device for each eye of a user. In such a system, sharing load between devices can be important to battery consumption. Both devices may be important (e.g., or necessary) for the functioning of the system. If one devices battery goes out before the other, both may no longer able to function. The present methods and systems can account for drains on the power supply when making decisions on load balancing. Drains on the power can comprise, for example, numerical processing energy consumption and the communication system's consumption.

Load balancing can be utilized for any operation performed by an ophthalmic device, such as a filtering operation, calculation, communication operation, and/or the like. For example, calculations are used to make a decision regarding accommodation, especially in the context of a vergence accommodation method. Load balancing can be utilized in the determination of which ophthalmic device (e.g., of a pair of ophthalmic devices disposed in or on a user eyes) is going to perform the computation and how the information is shared.

As an illustration, because everyone's eyes are a bit different, (e.g., pupil spacing and location, lens-on-eye position, etc.), even at a fixed close distance, initial vergence angles will differ from patient to patient. It is important once ophthalmic devices (e.g., lenses) are placed in or on the eye to calibrate what the initial vergence angle is, so that differences in this angle can be assessed while in service. This value can be used for subsequent calibration calculations. Load balancing can be used to in both the calibration process as well as other processes disclosed herein, such as customization of accommodation threshold, another other calculations related to eye gaze, accommodation, vergence (e.g., convergence, divergence), and/or the like.

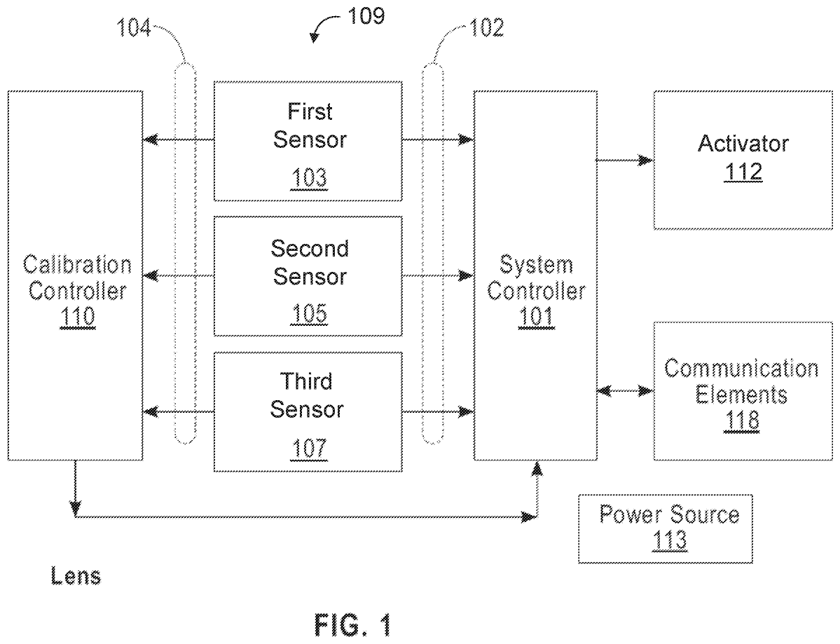

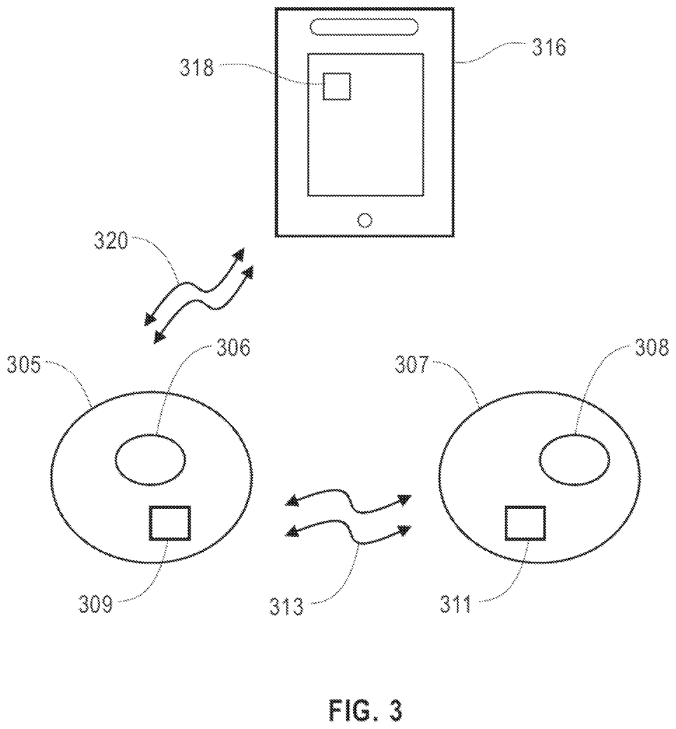

Now referring to FIG. 1, an exemplary implementation shows a system (e.g., sensor system) according to an embodiment of the present disclosure. The system can be disposed in or on an ophthalmic device. The ophthalmic device can comprise a contact lens or an implantable lens, or a combination of both. The ophthalmic device can be configured to be disposed adjacent an eye of a user. Adjacent to the eye may comprise disposed on a surface of the eye, in contact with the eye, resting on the eye, supported by the eye, disposed in a liquid on a surface of the eye, and/or the like. The contact lens comprises a soft or hybrid contact lens. The ophthalmic device can be part of a system of at least two ophthalmic devices, as shown in FIG. 3.

A system controller 101 controls an activator 112 (e.g., lens activator) that changes the adaptive optics/powered lens (see FIG. 3) to control the ability to see both near and far items in focus. The system controller 101 may comprise a processor, memory, and/or the like. The system controller 101 (e.g., the processor) may be operably coupled to a sensor element 109. The system controller 101 may receive signals 102 (e.g., data signals, control signals) from the sensor system 109.

The sensor element 109 can comprise a plurality of sensors (103, 105 and 107). Examples of sensors can comprise a multidimensional sensor, a capacitive sensor, an impedance sensor, an accelerometer, a temperature sensor, a displacement sensor, a neuromuscular sensor, an electromyography sensor, a magnetomyography sensor, a phonomyography, or a combination thereof. The plurality of sensors (103, 105 and 107) can comprise a lid position sensor, a blink detection sensor, a gaze sensor, a divergence level sensor, an accommodation level sensor, a light sensor, a body chemistry sensor, neuromuscular sensor, or a combination thereof. The plurality of sensors (103, 105 and 107) can comprise one or more contacts configured to make direct contact with tear film of an eye of the user.

As an illustration, the plurality of sensors (103, 105 and 107) can comprise a first sensor 103, such as a first multidimensional sensor that includes an X-axis accelerometer. The plurality of sensors (103, 105 and 107) can comprise a second sensor 105, such as a second multidimensional sensor that includes a Y-axis accelerometer. The plurality of sensors (103, 105 and 107) can comprise a third sensor 107, such as a third multidimensional sensor that includes a Z-axis accelerometer. The plurality of sensors (103, 105 and 107) further provide calibration signals 105 to a calibration controller 110. The calibration controller 110 conducts a calibration sequence based on the calibration signals from the plurality of multidimensional sensors (103, 105 and 107) as a result of user actions which is sensed by the plurality of multidimensional sensors (103, 105 and 107) and provides calibration control signals to the system controller 101. The system controller 101 further receives from and supplies signals to communication elements 118. Communication elements 118 allow for communications between user lens and other devices such a near-by smartphone. A power source 113 supplies power to all of the above system elements. The power source can comprise a battery. The power sources may be either a fixed power supply, wireless charging system, or may be comprised of rechargeable power supply elements. Further functionality of the above embedded elements is described herein.

As another embodiment, the three axis accelerometers can be replaced by a three-axis magnetometer. Calibration would be similar because each axis would potentially require calibration at each extreme of each axis.

In the context of using sensors to determine vergence, specifically accelerometers, there are opportunities to calibrate. Offsets, due to the micro-electromechanical systems (MEMS) and/or due to the electronics, mounting on the interposer, etc. can cause variations with the algorithms and thus cause some errors in the measurement of vergence. In addition, human anatomy from person to person, is different. For instance, eye to eye space can vary from 50 to 70 mm and can cause a change in trigger points based on eye spacing alone. So there is a need to take some of these variables out of the measurement, thus calibration and customization performed by the current embodiment when the lens are on the user. This serves to improve the user experience by both adding the preferences of the user and to reduce the dependencies of the above-mentioned variations.

The plurality of sensors (103, 105 and 107) can measure acceleration both from quick movements and from gravity (9.81 m/s²). The plurality of sensors (103, 105 and 107) usually produce a code that is in units of gravity (g). The determination of vergence depends on the measurement of gravity to determine position, but other methods may depend on the acceleration of the eye. There are going to be differences and inaccuracies that will require base calibration before use calibration.

The current embodiment uses three sensors on each ophthalmic device. However, calibration may be done using two sensors, e.g., the first sensor 103 (e.g., X-axis accelerometer) and the second sensor 105 (e.g., Y-axis accelerometer). In either embodiment, each accelerometer has a full scale plus, full scale minus, and zero position. The errors could be offset, linearity, and slope errors. A full calibration would calibrate to correct all three error sources for all of axes sensors being used.

One way to calibrate the sensors is to move them such that each axis is completely perpendicular with gravity, thus reading 1 g. Then the sensor would be turned 180 degrees and it should read −1 g. From two points, the slope and intercept can be calculated and used to calibrate. This is repeated for the other two sensors. This is an exhaustive way of calibrating the sensors and thus calibrating the vergence detection system.

Another way is to reduce the calibrate effort for the lens is to have the wearer do just one or two steps. One way is to have the wearer look forward, parallel to the floor, at a distance wall. Measurements taken at this time can be used to determine the offset of each axis. Determining the offset for each axis in the area where the user will spend most of the time provides a greater benefit to maintain accuracy.

Given that everyone is a little different, customizable features can prove a better user experience for all users than a one size fits all approach. When using the lens with just two modes, accommodation and gaze, then the point where this is a switch from gaze to accommodation one can have several parameters in addition to the switching threshold that would affect the user experience.

The threshold going from gaze to accommodation is depended on the user, the user's eye condition, the magnification of the lens, and the tasks. For reading, the distance between the eye and book is about 30 cm, where computer usage is about 50 cm. A threshold set for 30 cm wouldn't work well for computer work, but 50 cm would work for both. However, this longer threshold could be problematic for other tasks by activating too early, depending on the magnification and the user's own eye condition. Thus, the ability to alter this threshold, both when the lens is first inserted and at any time afterwards as different circumstances could require different threshold points, provides the user customization to improve visibility, comfort and possibly safety. Even having several present thresholds is possible and practical, where the user would choose using the interfaces described here to select a different threshold. In addition, the user could alter the threshold or other parameters by re-calibrating per the embodiments of the present disclosure as described hereafter.

Still referring to FIG. 1, switching from gaze to accommodation, the system uses the threshold as the activation point. However, going from accommodation to gaze the threshold is shifted to a greater distance, which is called hysteresis. Accounting for hysteresis is added in order to prevent uncertainty when the user is just at the threshold and there are small head movements which may cause it to switch from gaze to accommodation to gaze, etc. Most likely, the user will be looking at a distant target when he wants to switch, so the changing of the threshold is acceptable. The hysteresis value can be determined in several ways: one, the doctor fitting the lenses can change it, two, the user can change this value via a lens interface, and three, an adaptive algorithm can adjust it based on the habits of the user

Custom Modes are common now in cars, i.e. sport, economy, etc. which allow the user to pick a mode based on anticipated activity where the system alters key parameters to provide the best experience. Custom Modes are also integrated into the lens of the current embodiments. Calibration and customization settings can be optimized for a given mode of operation. If the user is working in the office, it is likely that the user will need to go between states (gaze and accommodation), or even between two different vergence distances because of the nature of the tasks. Changes in the threshold, hysteresis, noise immunity, and possible head positions would occur to provide quicker transitions, possible intermediate vergence positions, and optimization for computer tasks, as well as, tasks that there is a lot if switching between gaze and accommodation. Thus, options to switch the lens into different modes to optimize the lens operation can provide an enhanced user experience. Furthermore, in an “Exercise” mode, the noise filtering is increased to prevent false triggering and additional duration of positive signal is required before switching to prevent false switching of the lens being triggered by stray glances while running. A “Driving” mode might have the lens being configured for distant use or on a manual override only. Of course, various other modes that could be derived as part of the embodiments of the present disclosure.

In today's world, the smart phone is becoming a person's personal communications, library, payment device, and connection to the world. Apps for the smartphone cover many areas and are widely used. One possible way to interact with the lens of the present disclosure is to use a phone app. The app could provide ease of use where written language instructions are used and the user can interact with the app providing clear instructions, information, and feedback. Voice activation options may also be included. For instance, the app provides the prompting for the sensor calibrations by instructing the user to look forward and prompting the user to acknowledge the process start. The app could provide feedback to the user to improve the calibration and instruct the user what to do if the calibration is not accurate enough for optimal operation. This would enhance the user experience.

Additional indicators, if the smart phone was not available, can be simple responses from the system to indicate start of a calibration cycle, successful completion, and unsuccessful completion. Methods to indicate operation include, but are not limited to, blinking lights, vibrating haptics drivers, and activating the lens. Various patterns of activation of these methods could be interpreted by the user to understand the status of the lens. The user can use various methods to signal the lens that he/she is ready to start or other acknowledgements. For instance, the lens could be opened and inserted into the eyes awaiting a command. Blinks or even closing one's eyes could start the process. The lens then would signal the user that it is starting and then when it finishes. If the lens requires a follow-up, it signals the user and the user signals back with a blink or eye closing.

The system controller 101 can be configured to perform a load balancing procedure. For example, the system can comprise at least two ophthalmic devices, as shown later in FIG. 3. For purposes of illustration multiple ophthalmic devices are described, one or more (or each) of which can be an ophthalmic device as shown in FIG. 1. For example, a first ophthalmic device can be configured to be disposed adjacent a first eye of a user. As illustrated in FIG. 1, the first ophthalmic device can comprise a first sensor system. The first sensor system can comprise a first sensor and a first processor operably connected to the first sensor. A second ophthalmic device can be configured to be disposed adjacent a second eye of the user. The second ophthalmic device can comprise a second sensor system. The second sensor system can comprise a second sensor and a second processor operably connected to the second sensor.

The load balancing procedure can comprise any combination of at least two processing modes. The at least two processing modes can comprise a primary mode (e.g., full processing mode). While in primary mode, the ophthalmic device can receive data from one or more other ophthalmic devices, process the data, transmit an output of the processing, a combination thereof, and/or the like. The at least two processing modes can comprise a secondary mode (e.g., low power mode, partial processing mode, drone mode). While in the secondary mode, the ophthalmic device can be configured to receive and/or transmit data to another ophthalmic device that is operating in primary mode. While in secondary mode, the ophthalmic device can also implement instructions (e.g., adjust lens, modify power levels, change characteristic of the user, such as eye gaze, eye vergence, accommodation parameters) from another ophthalmic device.

In a dual-primary configuration, at least two ophthalmic devices can be configured to operate (e.g., simultaneously) in a primary mode. The at least two ophthalmic devices can be configured to each receive data from each other and perform the same or different processing based on the data. For example, a time period can be determined (e.g., based on a schedule, based on a synchronized clock, via a message from another ophthalmic device). During the time period, each of the at least two ophthalmic devices can perform a processing cycle. The processing cycle can comprise receiving data, processing the data, transmitting the data, a combination thereof, and/or the like. The received data can comprise an output of processing (e.g., by a different ophthalmic device) during a prior time period (e.g., a prior processing cycle). As a further explanation, the first processor can be configured to receive first data from the second sensor system. The second processor can determine a first characteristic of the user during a time period based on at least the first data. Example characteristics of the user are described further herein. The second processor can be configured to receive second data from the first sensor system. The second processor can determine a second characteristic of the user during the time period based on at least the second data. The first characteristic can be the same as the second characteristic. For example, both the second processor and the first processor can apply the same predefined function to the same or similar data. The first characteristic can be different from the second characteristic. For example, both the second processor and the first processor can apply the same predefined function or different functions to the same data, similar data, or different data.

In a primary-secondary configuration, one of the ophthalmic devices can operate in a primary mode while the other of the ophthalmic devices operates in a secondary mode. The ophthalmic devices can switch between primary mode and secondary mode. As a further explanation, the first processor can be configured to switch between the primary mode and the secondary mode to balance a processing load between the first ophthalmic device and the second ophthalmic device. For example, the first processor can be configured to operate in the primary mode while a second processor of the second ophthalmic device operates in a corresponding secondary mode. The first processor can be configured to operate in the secondary mode while the second processor of the second ophthalmic device operates in a corresponding primary mode.

As a further explanation, during the primary mode, the first processor can be configured to receive first data from one or more of the first sensor and a second sensor system disposed in a second ophthalmic device, determine a first characteristic of the user based on at least the first data, transmit the first characteristic of the user to the second sensor system, a combination thereof, and/or the like. During the secondary mode, the first processor can be configured to transmit second data to the second sensor system and/or receive a second characteristic of the user from the second sensor system. The second sensor system can determine the second characteristic of the user based on at least the second data. The first characteristic can be the same as the second characteristic. The first characteristic and/or second characteristic of the user can be determined based on a predefined function. For example, both the second processor and the first processor can apply the predefined function to the same or similar data. The first characteristic can be different from the second characteristic. For example, both the second processor and the first processor can apply the same predefined function or different functions to the same data, similar data, or different data.

Switching between primary mode and secondary mode can be performed based on a load balancing constraint. The load balancing constraint can specify that switching between modes occurs after a predefined number of processing cycles (e.g., 1, 2, 5, 10, 100, 100, or any other number), after a predefined amount of energy is consumed (e.g., by the device in the primary mode), after a predefined amount of battery life remains, after performing a predefined sequence of operations, a combination thereof, and/or the like.

To help quantify this concept, the following definitions are proposed: 1) a full calculation consumes 1 CPU energy unit and 2) a communication (TX and/or RX) cycle consumes 1 COM energy unit. The dual-primary configuration and the primary-secondary configuration are illustrated as follows via these definitions.

In the dual-primary configuration, a first ophthalmic device can communicate with the second ophthalmic device to transmit data from the first ophthalmic device. The second ophthalmic device can also communicate with the first ophthalmic device to transmit data from the second ophthalmic device. Both the first ophthalmic device and the second ophthalmic device can calculate outputs using the same algorithm (e.g., predefined function). Both the first ophthalmic device and the second ophthalmic device can reach the same conclusion and act accordingly. The total energy consumption during a time period (e.g., a processing cycle) can be two COMs and two CPUs with each device consuming one CPU and one COM via the device's respective battery. Data can be shared between the ophthalmic devices. For example, all data in the system can located at both the first ophthalmic device and the second ophthalmic device. The data may be used (e.g., in some cases, required) for certain algorithms (e.g., functions, calculations) and filters. The energy usage can be represented as follows:

Total energy/cycle=2*CPU+2*COM

Device Energy/cycle=CPU+COM

The primary-secondary configuration can comprise selecting one ophthalmic device to perform calculations (e.g., for a current measurement) for multiple ophthalmic devices, and then switching (e.g., back and forth) which ophthalmic device performs the calculations. Communications between the ophthalmic devices can send data from the current secondary ophthalmic device (e.g., device not performing calculations) to the current primary ophthalmic device. In addition, communications can send instructions back to the second ophthalmic device after the calculation is complete. If a filter or other calculation requires previous information, then at least one set of data can be sent from the previous primary ophthalmic device to the present ophthalmic device. The energy usage for this scenario can be represented as follows:

Total energy/cycle=CPU+3*COM

Primary Device Energy/cycle=CPU+2*COM

Secondary Device Energy/cycle=1COM

Over time, the ophthalmic devices can switch between which device is in primary mode and which device is in secondary mode to balance out energy usage between the ophthalmic devices. For example, each of the ophthalmic devices may monitor energy usage and/or battery level. The energy usage and/or battery level may be communicated from one ophthalmic device to another. If the battery life drops below a threshold for one of the ophthalmic devices (e.g., the one in primary mode), the ophthalmic device may request that the other ophthalmic device (e.g., the one in secondary mode) take on a larger processing load (e.g., change to a primary mode).

By way of comparison, the dual-primary configuration may use more or less total energy than the primary-secondary configuration, depending on the relative cost between the CPU and COM unit energy. For example, if 1 COM=1 CPU, then the two methods use the same energy. If COM is less than CPU, then the other method would be better. In addition, the dual-primary configuration can maintain more data for filtering and may be simpler to implement in an already complicated system.

The processing of data performed while in primary mode can comprise any operation, such as filtering (e.g., filtering noise), determining a measurement, determining a characteristic of a user, a combination thereof, and/or the like. The characteristic (e.g., first characteristic, second characteristic) of the user can comprise an eye vergence parameter (e.g., a vergence angle), a calibration parameter (e.g., sensor calibration setting), an accommodation parameter (e.g., accommodation threshold), an eye gaze parameter, an indication of a medical condition (e.g., a predisposition, a disease) a trigger (e.g., gaze value, divergence angle, light level, blink sequence) related to the user (e.g., for entering a specialized operation mode, such as a custom mode), and/or any other calculation.

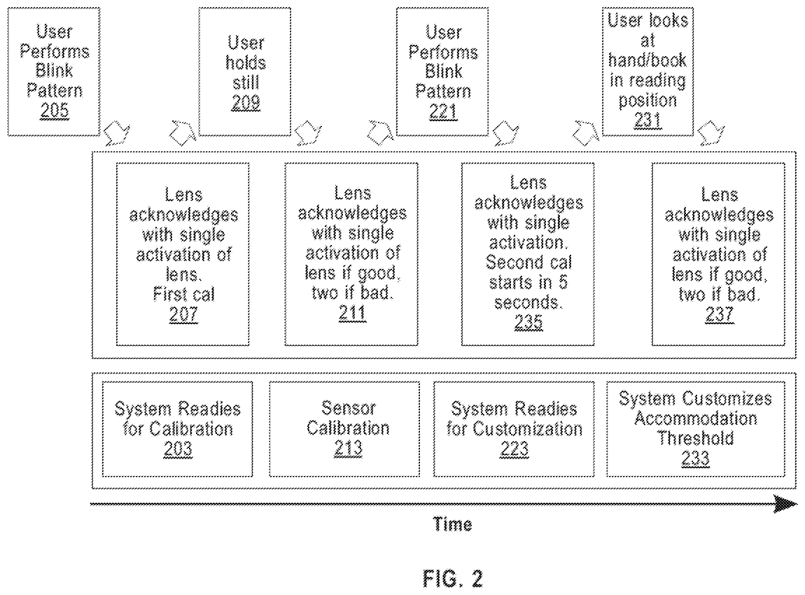

Referring to FIG. 2, one method according to an embodiment of the present disclosure is depicted. The process starts at an initial time (far left of the figure) and proceeds forward in time. Once the lens (see FIG. 3) are inserted, the system readies for calibration 203. The user performs a blink pattern 205. The lens acknowledges with a single activation of the lens 207 as part of a first calibration. The user holds still 209 as the system and the sensor calibration 213 starts. The lens acknowledges with a single activation of the lens if the first stage of calibration is good 211. If the initial calibration is bad, then the lens acknowledges with a double activation 211. If the calibration is bad, then the user must restart the calibration process 205. After the initial calibration, the system is ready for customization 223. The user conducts another blink pattern 221. The lens acknowledges with a single activation of the lens and a second calibration, customization, is started in some fixed time 235 as part the system customization accommodation threshold 233. The user then looks at either their hand or a book at reading position 231. The lens acknowledges with a single activation of the lens if the second stage of calibration customization is good 237. If the second stage of calibration customization is bad, then the user must restart the calibration customization process 221. Once the lens acknowledges with a single activation of the lens that the second stage of calibration customization is good 237 the system has the completed customization accommodation calibration and the lens are ready for full use by the user.

Other embodiments to customize the threshold can be accomplished. One way is to have the user's doctor determine the comfortable distance for the user by measuring the distance between the eyes of the patent, the typical distance for certain tasks, and then calculate the threshold. From there, using trial and error methods, determine the comfortable distance. Various thresholds can be programmed into the lens and the user can select the task appropriate threshold.

Another method is to allow the user to select his threshold himself. The lens can use the same system that it uses to measure the user's relative eye position to set the accommodation threshold. Where the user's preference of when to activate the extra lens power. There is an overlap where the user's eyes can accommodate unassisted to see adequately and where the user's eyes also can see adequately with the extra power when the lens is active. At what point to activate determined by user preference. Providing a means for the user to set this threshold, improves the comfort and utility of the lenses. The procedure follows this sequence:
The user prompts the system to start the sequence. Initially the system could prompt the user as a part of the initial calibration and customization;
The lenses are activated. The ability to achieve a comfortable reading position and distance requires the user to actually see the target, thus the lens are in the accommodation state;
The user focuses on a target which is at a representative distance while the system determines the distance based on the angles of the eyes by using the sensor information (accelerometers or magnetometers); after several measurements and noise reduction techniques the system calculates a threshold and indicates that it has finished,
The new threshold has been determined. A slight offset is subtracted to effectively place the threshold a little farther away, thus creating hysteresis. This is necessary to move the threshold slightly longer (angle slightly lower) in order to guarantee when the user is in the same position, the system will accommodate even with small head or body position differences; The value of this hysteresis could be altered by an algorithm that adapts to user habits. Also, the user could manually change the value if the desired by having the system prompt the user to move the focus target to a position that the user does not want the lenses to activate all the while focusing on the target. The system would deactivate the lenses and then determine this distance. The Hysteresis value is the difference in the deactivate distance and the activate distance. Lenses are now on dependent on the new threshold and hysteresis values

To have a good user experience, the user can receive confirmation that the system has completed any adjustments or customization. In addition, the system can be configured to determine if the user performed these tasks properly and if not, and then request that the user preforms the procedure again. Cases that prevent proper customization and adjustment may include excessive movement during measurement, head not straight, lens out of tolerance, etc. The interactive experience will have far less frustrated or unhappy users.

Feedback can be given through various means. Using a phone app provides the most flexibility with the screen, cpu, memory, internet connection, etc. The methods as discussed for calibration per the embodiments of the present disclosure can be done in conjunction with the use of a smartphone app with use of the communication elements as described in reference to FIG. 1 and with reference to FIG. 3 hereafter.

As a part of continual improvement for the lens, data for the ophthalmic devices can be collected and sent back to the manufacturer (anonymously) via the smartphone app to be used to improve the product. Collected data includes, but not limited to, accommodation cycles, errors, frequency that poor conditions occur, number of hours worn, user set threshold, etc.

Other methods to indicate operation include, but not limited to, blinking lights, vibrating haptics drivers, and activating the ophthalmic devices. Various patterns of activation of these methods could be interpreted by the user to understand the status of the ophthalmic device.

Referring now to FIG. 3, shown is another exemplary implementation according to an embodiment of the present disclosure in which sensing and communication may be used to communicate between a pair of ophthalmic devices (305, 307), such as contact lenses. Pupils (306, 308) are illustrated for viewing objects. The ophthalmic devices (305, 307) include embedded elements, such as those shown in FIG. 1. The embedded elements (309, 311) included for example 3-axis accelerometers/magnetometers, lens activators, calibration controller, a system controller, memory, power supply, and communication elements as is described in detail subsequently. A communication channel 313 between the two ophthalmic devices (305, 307) allows the embedded elements to conduct calibration between the ophthalmic devices (305, 307). Communication may also take place with an external device, for example, spectacle glasses, key fob, dedicated interface device, or a smartphone.

Communication between the two ophthalmic devices (305, 307) can be performed in order to implement a load balancing scheme. The ophthalmic devices (305, 307) can periodically communicate data, such as sensor data, output of calculations (e.g., characteristic of a user), parameter data (e.g., filters applied). Communication between the two ophthalmic devices (305, 307) can be periodically performed, such as a predefined number of times during a time period, according to specific schedule, in response to a triggering condition, and/or the like. In a dual-primary configuration, both ophthalmic devices (305, 307) can communicate outputs of calculations (e.g., characteristics of users), sensor data, and other data. In a primary-secondary configuration, the ophthalmic device in secondary mode can receive outputs of calculations from the ophthalmic device in primary mode. The ophthalmic device in secondary mode can transmit stored data (e.g., sensor data), data from a previous time period (e.g., processing cycle), and/or the like. After receiving data from the ophthalmic device in secondary mode, the ophthalmic device in primary mode can perform one or more calculations and provide the output back to the device in secondary mode.

As an example, communication between the ophthalmic devices (305, 307) can be important to detect proper calibration. Communication between the two ophthalmic devices (305, 307) may take the form of absolute or relative position, or may simply be a calibration of one lens to another if there is suspected eye movement. If a given ophthalmic device detects calibration different from the other ophthalmic device, it may activate a change in stage, for example, switching a variable-focus or variable power optic equipped contact lens to the near distance state to support reading. Other information useful for determining the desire to accommodate (focus near), for example, lid position and ciliary muscle activity, may also be transmitted over the communication channel 313. It should also be appreciated that communication over the channel 313 could comprise other signals sensed, detected, or determined by the embedded elements (309, 311) used for a variety of purposes, including vision correction or vision enhancement.

The communications channel (313) comprises, but not limited to, a set of radio transceivers, optical transceivers, or ultrasonic transceivers that provide the exchange of information between both lens and between the lenses and a device such as a smart phone, FOB, or other device used to send and receive information. The types of information include, but are not limited to, current sensor readings showing position, the results of system controller computation, synchronization of threshold and activation. In addition, the device or smart phone could upload settings, sent sequencing signals for the various calibrations, and receive status and error information from the lenses.

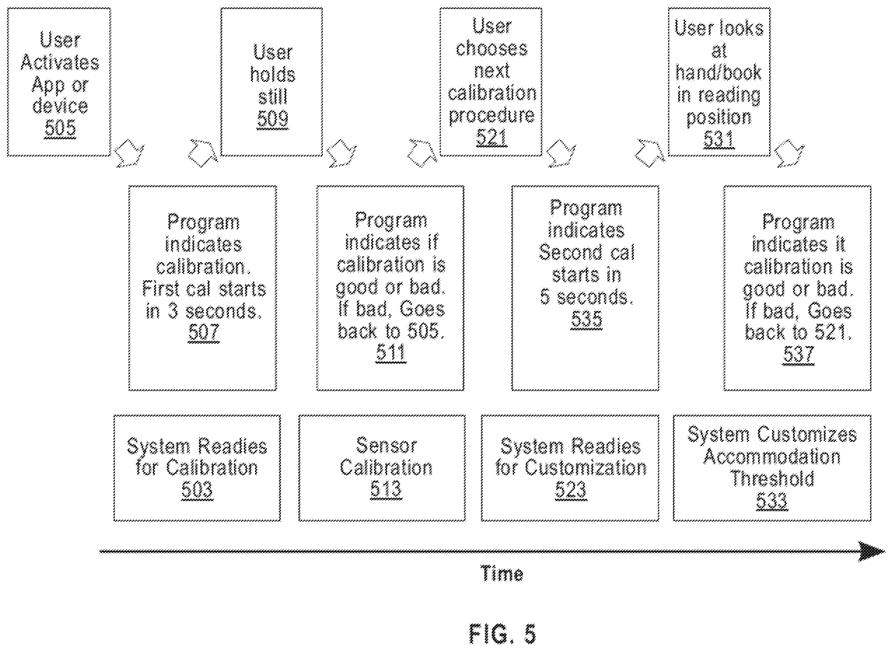

Still referring to FIG. 3, the ophthalmic devices (305, 307) further communicate with a smart phone (316) or other external communication device. Specifically, an app 318 on the smart phone (316) communicates to the ophthalmic devices (305, 307) via a communication channel (320). The functionally of the app (318) follows the process as outlined with referenced to FIG. 5 (described hereafter) and instructs the user when to perform the required eye movements. In addition, the device or smart phone (316) could upload settings, sent sequencing signals for the various calibrations, and receive status and error information from the contact lenses (305, 307).

Referring to FIG. 5, another method according to an embodiment of the present disclosure is depicted. The process starts at an initial time (far left of the figure) and proceeds forward in time. Once the ophthalmic devices (see FIG. 3) are inserted, the system readies for calibration 503. User activates App or device 205. The app program indicates calibration and the first calibration starts in 3 seconds 507 as part of a first calibration. The user holds still 509 as the system and the sensor calibration 513 starts. The program indicates if calibration is good or bad 511. If calibration is bad the program restarts and goes back (to step 505) 511. After the initial calibration, the system is ready for customization 523. The user chooses the next calibration procedure 521. The program indicates the second calibration will start in 5 seconds 535 as part the system customization accommodation threshold 533. The user then looks at either their hand or a book at reading position 531. The program determines if second stage of calibration customization is good 537. If the second stage of calibration customization is bad, then the user must restart the calibration customization process 521. Once the program acknowledges that the second stage of calibration customization is good 537 the system has the completed customization accommodation calibration and the lenses are ready for full use by the user. As a non-limiting example, the calibration process may leverage load sharing as described herein. For example, one of a pair of lenses may be used for the calibration process and may then transmit calibration settings to the second of the pair of lenses for calibration of both lenses. As such, the CPU load may be minimized in the second lens as compared to the CPU load in the first lens for calibration. Further load sharing methods may be used.

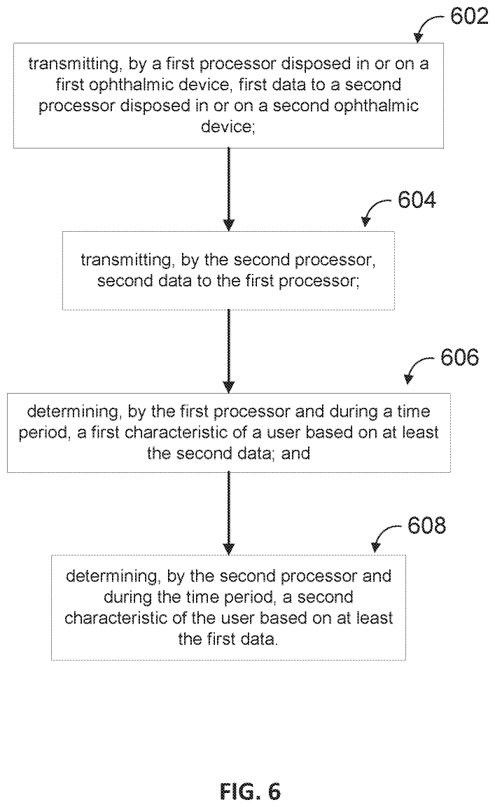

As an example, FIG. 6 illustrates a method according to aspects of the present disclosure. In step 602, a first processor disposed in or on a first ophthalmic device may transmit first data to a second processor disposed in or on a second ophthalmic device. The first ophthalmic device may comprise a first battery and the second ophthalmic device comprises a second battery. In certain aspects, the first data is from a first sensor disposed within the first ophthalmic device, and wherein the first sensor comprises a capacitive sensor, an impedance sensor, an accelerometer, a temperature sensor, a displacement sensor, a neuromuscular sensor, an electromyography sensor, a magnetomyography sensor, a phonomyography, or a combination thereof. In certain aspects, the first data is from a first sensor disposed within the first ophthalmic device, and wherein the first sensor comprises a lid position sensor, a blink detection sensor, a gaze sensor, divergence level sensor, an accommodation level sensor, a light sensor, a body chemistry sensor, neuromuscular sensor, or a combination thereof. In certain aspects, the first data is from a first sensor disposed within the first ophthalmic device, wherein the first sensor comprises one or more contacts configured to make direct contact with tear film of an eye of the user. In step 604, the second processor may transmit second data to the first processor. In certain aspects, the second data is from a second sensor disposed within the second ophthalmic device, and wherein the second sensor comprises a capacitive sensor, an impedance sensor, an accelerometer, a temperature sensor, a displacement sensor, a neuromuscular sensor, an electromyography sensor, a magnetomyography sensor, a phonomyography, or a combination thereof. In certain aspects, the second data is from a second sensor disposed within the second ophthalmic device, and wherein the second sensor comprises a lid position sensor, a blink detection sensor, a gaze sensor, divergence level sensor, an accommodation level sensor, a light sensor, a body chemistry sensor, neuromuscular sensor, or a combination thereof. In certain aspects, the second data is from a second sensor disposed within the second ophthalmic device, wherein the second sensor comprises one or more contacts configured to make direct contact with tear film of an eye of the user. In step 606, the first processor may determine, during a time period, a first characteristic of a user based on at least the second data. The time period may comprise a time period to complete a single processing cycle. In certain aspects, determining, by the first processor and during the time period, the first characteristic of the user based on at least the second data consumes an energy value within a threshold of equivalence to an energy value consumed in determining, by the second processor and during a time period, the second characteristic of a user based on at least the first data. For example, the first processor and second processor may have the same design and run the same process but may differ in energy consumption due to variability, such as manufacturing process variability, operating condition variability, and/or the like. The threshold equivalence may be a threshold associated with the allowed variability of the first processor and/or second processor. The threshold may not be explicitly defined or stored as a value but may be understood as a general range of expected variability for the ophthalmic devices. In certain aspects, determining, by the first processor and during the time period, the first characteristic of the user based on at least the second data and determining, by the second processor and during the time period, the second characteristic of the user based on at least the first data are both performed based on a predefined function. In step 608, the second processor may determine, during the time period, a second characteristic of the user based on at least the first data. One or more of the first characteristic and the second characteristic of the user may comprise an accommodation parameter. One or more of the first characteristic and the second characteristic of the user comprises an eye vergence parameter. One or more of the first characteristic and the second characteristic of the user comprises an eye gaze parameter. As an example, the characteristic of the user comprises an indication of a medical condition, such as an indication of disease. The first and/or second ophthalmic device may comprises a contact lens or an implantable lens, or a combination of both. The contact lens may comprise a soft or hybrid contact lens. In certain aspects, determining the characteristic of the user is performed by both the first processor and the second processor as part of a load balancing scheme that balances energy consumption between the first ophthalmic device and the second ophthalmic device.

It is important to note that the above described elements may be realized in hardware, in software or in a combination of hardware and software. In addition, the communication channel may comprise any include various forms of wireless communications. The wireless communication channel may be configured for high frequency electromagnetic signals, low frequency electromagnetic signals, visible light signals, infrared light signals, and ultrasonic modulated signals. The wireless channel may further be used to supply power to the internal embedded power source acting as rechargeable power means.

The present disclosure may be a system, a method, and/or a computer program product. The computer program product being used by a controller for causing the controller to carry out aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An ophthalmic system comprising:
a first ophthalmic device configured to be disposed adjacent an eye of a user; and
a first sensor system disposed in or on the first ophthalmic device, the first sensor system comprising a first sensor and a first processor operably connected to the first sensor and configured to alternate between a primary mode and a secondary mode,
wherein, during the primary mode, the first processor is configured to receive first data from one or more of the first sensor and a second sensor system disposed in a second ophthalmic device, determine a first characteristic of the user based on at least the first data, and transmit the first characteristic of the user to the second sensor system, and
wherein, during the secondary mode, the first processor is configured to transmit second data to the second sensor system and receive a second characteristic of the user from the second sensor system, wherein the second sensor system determines the second characteristic of the user based on at least the second data.

2. The ophthalmic system according to claim 1, wherein the first ophthalmic device comprises a contact lens.

3. The ophthalmic system according to claim 2, wherein the contact lens comprises a soft or hybrid contact lens.

4. The ophthalmic system according to claim 1, wherein the first ophthalmic device comprises a contact lens or an implantable lens, or a combination of both.

5. The ophthalmic system according to claim 1, wherein determining the first characteristic of the user by the first processor is performed based on a predefined function.

6. The ophthalmic system according to claim 1, wherein the first characteristic of the user comprises an accommodation parameter.

7. The ophthalmic system according to claim 1, wherein the first characteristic of the user comprises an eye vergence parameter.

8. The ophthalmic system according to claim 1, wherein the first characteristic of the user comprises an eye gaze parameter.

9. The ophthalmic system according to claim 1, wherein the first sensor comprises a capacitive sensor, an impedance sensor, an accelerometer, a temperature sensor, a displacement sensor, a neuromuscular sensor, an electromyography sensor, a magnetomyography sensor, a phonomyography, or a combination thereof.

10. The ophthalmic system according to claim 1, wherein the first sensor comprises a lid position sensor, a blink detection sensor, a gaze sensor, divergence level sensor, an accommodation level sensor, a light sensor, a body chemistry sensor, neuromuscular sensor, or a combination thereof.

11. The ophthalmic system according to claim 1, wherein the first ophthalmic device comprises a first battery and the second ophthalmic device comprises a second battery.

12. The ophthalmic system according to claim 1, wherein the first sensor comprises one or more contacts configured to make direct contact with tear film of an eye of the user.

13. The ophthalmic system according to claim 1, wherein the first characteristic of the user comprises an indication of a medical condition.

14. The ophthalmic system according to claim 13, wherein the medical condition comprises an indication of disease.

15. The ophthalmic system according to claim 1, wherein the first processor is configured to switch between the primary mode and the secondary mode to balance a processing load between the first ophthalmic device and the second ophthalmic device.

16. The ophthalmic system according to claim 1, wherein the first processor is configured to operate in the primary mode while a second processor of the second ophthalmic device operates in a corresponding secondary mode, and wherein the first processor is configured to operate in the secondary mode while the second processor of the second ophthalmic device operates in a corresponding primary mode.

17. The ophthalmic system according to claim 1, wherein the first characteristic is the same as the second characteristic.

18. The ophthalmic system according to claim 1, wherein the first characteristic is different from the second characteristic.

19. An ophthalmic system comprising:
a first ophthalmic device configured to be disposed adjacent a first eye of a user, the first ophthalmic device comprising a first sensor system, the first sensor system comprising a first sensor and a first processor operably connected to the first sensor; and
a second ophthalmic device configured to be disposed adjacent a second eye of the user, the second ophthalmic device comprising a second sensor system, the second sensor system comprising a second sensor and a second processor operably connected to the second sensor,
wherein the first processor is configured to receive first data from the second sensor system and determine a first characteristic of the user during a time period based on at least the first data, and wherein the second processor is configured to receive second data from the first sensor system and determine a second characteristic of the user during the time period based on at least the second data.

20. The ophthalmic system according to claim 19, wherein the first ophthalmic device comprises a contact lens.

21. The ophthalmic system according to claim 20, wherein the contact lens comprises a soft or hybrid contact lens.

22. The ophthalmic system according to claim 19, wherein the first ophthalmic device comprises a contact lens or an implantable lens, or a combination of both.

23. The ophthalmic system according to claim 19, wherein determining the first characteristic of the user by the first processor consumes an energy value within a threshold equivalence to an energy value consumed in determining the second characteristic by the second processor.

24. The ophthalmic system according to claim 19, wherein determining the first characteristic of the user by the first processor and determining the second characteristic of the user by the second processor are both performed based on a predefined function.

25. The ophthalmic system according to claim 19, wherein the first characteristic of the user comprises an accommodation parameter.

26. The ophthalmic system according to claim 19, wherein the first characteristic of the user comprises an eye vergence parameter.

27. The ophthalmic system according to claim 19, wherein the first characteristic of the user comprises an eye gaze parameter.

28. The ophthalmic system according to claim 19, wherein determining the first characteristic of the user by the first processor and determining the second characteristic of the user by the second processor is performed as part of a load balancing scheme that balances energy consumption between the first ophthalmic device and the second ophthalmic device.

29. The ophthalmic system according to claim 19, wherein the first sensor comprises a capacitive sensor, an impedance sensor, an accelerometer, a temperature sensor, a displacement sensor, a neuromuscular sensor, an electromyography sensor, a magnetomyography sensor, a phonomyography, or a combination thereof.

30. The ophthalmic system according to claim 19, wherein the first sensor comprises a lid position sensor, a blink detection sensor, a gaze sensor, divergence level sensor, an accommodation level sensor, a light sensor, a body chemistry sensor, neuromuscular sensor, or a combination thereof.

31. The ophthalmic system according to claim 19, wherein the first ophthalmic device comprises a first battery and the second ophthalmic device comprises a second battery.

32. The ophthalmic system according to claim 19, wherein the first sensor comprises one or more contacts configured to make direct contact with tear film of an eye of the user.

33. The ophthalmic system according to claim 19, wherein the first characteristic of the user comprises an indication of a medical condition.

34. The ophthalmic system according to claim 33, wherein the medical condition comprises an indication of disease.

35. The ophthalmic system according to claim 20, wherein the time period comprises a time period to complete a single processing cycle.

36. The ophthalmic system according to claim 20, wherein the first characteristic is the same as the second characteristic.

37. The ophthalmic system according to claim 20, wherein the first characteristic is different from the second characteristic.

38. A method for balancing load in an ophthalmic device, the method comprising:
transmitting, by a first processor disposed in or on a first ophthalmic device, first data to a second processor disposed in or on a second ophthalmic device;
transmitting, by the second processor, second data to the first processor;
determining, by the first processor and during a time period, a first characteristic of a user based on at least the second data; and
determining, by the second processor and during the time period, a second characteristic of the user based on at least the first data.

39. The method according to claim 38, wherein the first ophthalmic device comprises a contact lens.

40. The method according to claim 39, wherein the contact lens comprises a soft or hybrid contact lens.

41. The method according to claim 38, wherein the first ophthalmic device comprises a contact lens or an implantable lens, or a combination of both.

42. The method according to claim 38, wherein determining, by the first processor and during the time period, the first characteristic of the user based on at least the second data consumes an energy value within a threshold equivalence to an energy value consumed in determining, by the second processor and during a time period, the second characteristic of a user based on at least the first data.

43. The method according to claim 38, wherein determining, by the first processor and during the time period, the first characteristic of the user based on at least the second data and determining, by the second processor and during the time period, the second characteristic of the user based on at least the first data are both performed based on a predefined function.

44. The method according to claim 38, wherein one or more of the first characteristic and the second characteristic of the user comprises an accommodation parameter.

45. The method according to claim 38, wherein one or more of the first characteristic and the second characteristic of the user comprises an eye vergence parameter.

46. The method according to claim 38, wherein one or more of the first characteristic and the second characteristic of the user comprises an eye gaze parameter.

47. The method according to claim 38, wherein the determining the characteristic of the user is performed by both the first processor and the second processor as part of a load balancing scheme that balances energy consumption between the first ophthalmic device and the second ophthalmic device.

48. The method according to claim 38, wherein the first data is from a first sensor disposed within the first ophthalmic device, and wherein the first sensor comprises a capacitive sensor, an impedance sensor, an accelerometer, a temperature sensor, a displacement sensor, a neuromuscular sensor, an electromyography sensor, a magnetomyography sensor, a phonomyography, or a combination thereof.

49. The method according to claim 38, wherein the first data is from a first sensor disposed within the first ophthalmic device, and wherein the first sensor comprises a lid position sensor, a blink detection sensor, a gaze sensor, divergence level sensor, an accommodation level sensor, a light sensor, a body chemistry sensor, neuromuscular sensor, or a combination thereof.

50. The method according to claim 38, wherein the first ophthalmic device comprises a first battery and the second ophthalmic device comprises a second battery.

51. The method according to claim 38, wherein the first data is from a first sensor disposed within the first ophthalmic device, wherein the first sensor comprises one or more contacts configured to make direct contact with tear film of an eye of the user.

52. The method according to claim 38, wherein the characteristic of the user comprises an indication of a medical condition.

53. The method according to claim 52, wherein the medical condition comprises an indication of disease.

54. The method according to claim 38, wherein the time period comprises a time period to complete a single processing cycle.

55. The method according to claim 38, wherein the first ophthalmic device is disposed in a first eye of the user and the second ophthalmic device is disposed in a second eye of the user.

56. The method according to claim 38, wherein the first characteristic is the same as the second characteristic.

57. The method according to claim 38, wherein the first characteristic is different from the second characteristic.
